(19) United States
(12) Patent Application Publication
(10) Pub. No.: US 2022/0079488 A1
(43) Pub. Date: Mar. 17, 2022

(54) PORTABLE NECK STRENGTH TESTING DEVICE

(71) Applicants: Martin Daniel Furtado, Thunder Bay (CA); Antonio Duarte Furtado, L'lle Bizard (CA)

(72) Inventors: Martin Daniel Furtado, Thunder Bay (CA); Antonio Duarte Furtado, L'lle Bizard (CA)

(21) Appl. No.: 17/476,119

(22) Filed: Sep. 15, 2021

Related U.S. Application Data
(60) Provisional application No. 63/078,843, filed on Sep. 15, 2020.

(51) Int. Cl. A61B 5/22; A61B 5/11; A61B 5/00

(52) U.S. Cl. CPC A61B 5/224; A61B 5/1114; A61B 2090/066; A61B 5/6823; A61B 5/6814

(57) ABSTRACT

A portable neck strength testing device includes a support member securable to the body of a subject, a measurement unit coupled to the support member and a head mount adjustably connected to the measurement unit. The head mount includes a first portion configured to engage the head of the subject and the head mount is displaceable in response to motion of the first portion. The measurement unit is configured to detect displacement of the head mount and measure therefrom at least a force or a torque exerted on the head mount.

### CROSS-REFERENCE TO RELATED PATENT APPLICATION

This application claims priority from U.S. Provisional Patent Application No. 63/078,843, filed on Sep. 15, 2020. The entire contents of U.S. Provisional Patent Application No. 63/078,843 is herein incorporated by reference for all purposes.

### FIELD OF THE DISCLOSURE

The present subject matter relates to portable devices and methods for measuring neck strength of a subject.

### INTRODUCTION

Concussions in youth sports continue to be an issue in today's society. The National Ambulatory Care Reporting System (NACRS) reported approximately 46 000 diagnosed concussions in 2016-2017 by hospital emergency departments for children and youth 5-19 years of age. Of the reported concussions, approximately 50% occurred during sports or recreation activities. The high incidence of concussions demands new intervention strategies to help decrease and/or prevent concussions for youth especially those participating in sports and recreation activities.

Although the importance of strengthening neck muscles has been recognized, existing neck strength measurement devices are often cumbersome or pose difficulties with stabilizing a subject during neck strength testing. For example, some devices offer methods of measuring neck flexion force that involve cumbersome stationary devices requiring external attachments or hand-held dynamometer force meters that require practitioner/operator stabilization. Stationary devices present limited access for testing, requiring subjects to travel to a testing laboratory to record neck flexion strength measurements. Hand-held dynamometer devices pose challenges for those administering the test associated with stabilization of the handheld measurement device during the testing procedure. Hand-held devices may also pose challenges for the subject being tested as they are not capable of providing stabilization of body parts not being tested during the testing procedure.

### SUMMARY OF THE DISCLOSURE

According to one aspect of the teachings herein, there is disclosed a portable neck strength testing device, comprising:
a support member securable to a body of a subject;
a measurement unit coupled to the support member;
head mount adjustably connected to the measurement unit,
wherein the head mount comprises a first portion configured to engage a head of the subject;
wherein the head mount is displaceable in response to motion of the first portion; and
wherein the measurement unit is configured to detect displacement of the head mount and measure therefrom at least a force or a torque exerted on the head mount.

For example, the support member may include:
one or more front legs for mounting the support member over the chest of the subject;
a shoulder section connected to the one or more front legs;
an adjustable back plate for mounting the support member over the back of the subject, wherein the back plate is connected to the shoulder section;
a back plate adjustment fastener located on the shoulder section and configured to adjust the back plate on the back of the subject to secure and stabilize the device on the upper body of the subject; and
a post connected to the shoulder section and extending vertically away from the back plate.

For example, the shoulder section may include a turntable and a base, wherein the front legs and the back plate are mounted on the turntable base, the post is mounted on the rotating turntable, and the rotating turntable is rotatable with respect to the turntable base.

The device may further include a removable torque measuring assembly for measuring neck rotation torque in either a left or right direction, wherein the torque measuring assembly includes a pair of arms extending away from the shoulder section, an overhead cross member mounted on the pair of arms, and a rotational torque measuring unit mounted on the overhead cross member and configured to engage the head of the subject for measuring the torque in response to rotation of the head.

The measurement unit may be adjustably mounted on the vertical post.

The measurement unit may comprise a force transducer, and the head mount may be adjustably connected to the force transducer for detecting and measuring a force exerted on the head mount.

In some cases, the force transducer is adjustable on the vertical post such that the force transducer and the head mount are positionable to align with the occipital tuberosity of the head of the subject.

The force transducer may be configured to measure neck flexion strength relative to the vertical post from motion of the head mount when the head mount engages the head of the subject.

The head mount may be displaceable in response to various motions of the first portion or the head mount, including front flexion, lateral flexion, extension, or a direction defined within a range of 0-360°.

The measurement unit may be configured to measure a maximum rotational torque applied by neck muscles of a subject when the head is turned to the left or right directions.

The support member may include a material including aluminum, plastic or carbon fiber.

According to one aspect, a method for measuring a force exerted by neck muscles of a subject includes:
securing a portable neck strength testing device to the body of the subject, the device having a support member secured to the subject, a measurement unit coupled to the support member and a head mount adjustably connected to the measurement unit;
engaging the head of the subject with the head mount; and
detecting, with the measurement unit, displacement of the head mount and measuring therefrom at least a force or a torque exerted on the head mount

Securing the device to the subject may include
mounting one or more front legs of the device over the chest of the subject, the one or more legs being connected to a shoulder section of the device;
mounting an adjustable back plate of the device over the back of the subject, the back plate being connected to the shoulder section of the device; and
adjusting, with a back plate adjustment fastener located on the shoulder section of the device, the back plate on the back of the subject to secure and stabilize the device on the upper body of the subject.

The method may further include measuring a force applied by neck muscles of the subject to the head mount with a force transducer connected to the head mount.

The method may further include adjusting the force transducer on a vertical post connected to the shoulder section and extending vertically away from the back plate such that the force transducer and the head mount are positionable to align with the occipital tuberosity of the head of the subject.

The method may further include measuring with the force transducer neck flexion strength relative to the vertical post from motion of the head mount when the head mount engages the head of the subject.

The method may further include measuring with the force transducer a maximum rotational torque applied by neck muscles of the subject when the head is turned to the left or right directions.

### BRIEF DESCRIPTION OF THE DRAWINGS

FIG. 1 shows a perspective view of a portable neck strength testing device according to one example;

FIG. 2 shows a side view of the device of FIG. 1;

FIG. 3 shows a perspective view of a portable neck strength testing device according to one example;

FIG. 4 is a perspective view of a portable neck strength testing device according to one example;

FIG. 5 shows a flow chart of a method for measuring neck strength according to one example.

FIG. 6 shows a flow chart of a method for measuring neck strength according to one example.

### DETAILED DESCRIPTION OF THE DISCLOSURE

It will be appreciated that, for simplicity and clarity of illustration, where considered appropriate, reference numerals may be repeated among the figures to indicate corresponding or analogous elements or steps. Numerous specific details are set forth in order to provide a thorough understanding of the exemplary embodiments of the subject matter described herein. However, it will be understood by those of ordinary skill in the art that the embodiments described herein may be practiced without these specific details. In other instances, well-known methods, procedures and components have not been described in detail so as not to obscure the present subject matter. Furthermore, this description is not to be considered as limiting the scope of the subject matter in any way but rather as illustrating the various embodiments.

The present subject matter relates to methods and portable devices for measuring neck strength. The device can include a force transducer means to measure the strength of the neck muscles of a subject, for example, when the neck is flexed forcefully to move the chin toward the top of the chest, when the neck is flexed forcefully to move the head in a direction from 0-360° (i.e. lateral flexion=90°, extension=180°, etc.), or when the head is torqued left or right.

The present subject matter may solve one or more of the problems associated with prior neck muscle strength measurement devices, such as by providing better and/or different ways of measuring the strength of the neck flexion muscles in an efficient and accurate manner. The present subject matter may allow neck flexion strength measurements to be made without the requirement of external attachments or practitioner/operator stabilization of the measuring device.

Accordingly, the teachings herein may provide a portable isometric neck strength testing device which functions independent of attachment to a fixed structure or connection to an operator to stabilize the device during testing procedures.

The teachings herein may provide a testing device for measuring neck strength during flexion of the neck in various directions including front flexion, lateral flexion, extension, or a direction defined within the range of 0-360°.

The teachings herein may provide a testing device that measures the maximum rotational torque applied by the neck muscles when the head is turned to the left or right directions.

The teachings herein may provide such a testing device which can be quickly and easily donned and removed from the body of the wearer.

One embodiment of the present subject matter relates to a portable device that is constructed of a rigid material such as aluminum, plastic, carbon fibre or other suitable material.

For example, the device can include a pair of front vertical legs which overlie the chest of the subject (i.e., a human being) and then bend over the shoulder in a horizontal shoulder section continue behind the head and then converge into a single posterior vertical post that extends vertically up away from the back of the head. A separate adjustable back plate section can overlie the back of the subject. The adjustable back plate can be removably connected to the shoulder portion of the device and secure the device on the subject being tested. A head mount including a strap can be attached to a measurement unit.

Furthermore, the embodiments of the present disclosure can include a method of fitting the device on subjects with chests of various shapes by employing adjustable hinges on the front legs near the top of each leg as they bend over the shoulder, thereby allowing the device to be properly balanced on the subject with the posterior vertical post in the vertical position.

The measurement unit can include a force transducer (e.g. force sensor, strain gauge, thin film sensor, etc.). For example, the force transducer can generate an output charge or voltage that is proportional to the force applied to the transducer. The output charge is then used to calculate a force applied to the force transducer to measure the strength of neck muscles of a subject.

The measuring unit can measure the strength of the neck muscles when the head mount engages a head of the subject. For example, measurements can be recorded by the force transducer when the neck is flexed to move the chin toward the top of the chest, when the neck is flexed to move the head in a direction from 0-360° (i.e. lateral flexion=90°, extension=180°, etc.), or when the head is torqued left or right. For example, a processor of the measuring unit can compute the strength of the neck muscles based on the output charge or voltage recorded by the force transducer.

The measuring unit can include a processor to transmit, via Bluetooth connectivity, collected and processed data to an external device, such as a smartphone, a personal computer or a server. For example, the measuring unit can have an input interface (e.g. a keyboard) that allows a user to enter data such as neck circumference, head circumference, neck length, age, gender, etc. In addition to force data recorded by the force transducer, the processor may use the user input data to compute the strength of the neck muscles of a subject.

For example, the processor may compare the user input data to a set of historical data (e.g. data collected for other subjects and/or circumstances pertaining to a particular subject) to establish a baseline for neck strength measurements. This may improve the accuracy of the measurements. The measuring unit can also have a screen or any other type of output interfaces to display collected or processed data.

The measurement unit can be an adjustable fixture on the posterior vertical post. This allows the force transducer to be moved vertically on the vertical post and secured to align the transducer of the measurement unit and the head strap positioned on the head to the occipital tuberosity of the wearer's head.

The operation of the device can include a method of lowering the device onto the subject being tested followed by adjustment of the back plate tight against the wearer's back, thereby allowing the body to be positioned so the parts of the body containing muscles which are not part of the measurement are stabilized. For example, the positioning of the device on the subject in this manner serves to isolate the neck muscles during testing which is a significant improvement to prior measurement devices.

For example, the embodiments of the present disclosure can serve as a first class lever where measurement of the neck muscle strength is made possible by using the muscles themselves to provide the load on the neck as it is flexed. Accordingly, no external attachment or practitioner/operator stabilization may be required by the device to perform a measurement. The embodiments of the present disclosure render portability thereby allowing measurements to be made at remote sites.

Furthermore, the embodiments of the present disclosure may also be used in a fixed position by employing a mounting means to fix the device to a support structure. The testing procedure may include but is not limited to the subject being instructed to stand upright while the device is fitted, to perform three practice neck flexion movements to become familiar with the flexion motion direction while the device is secured to the upper body, and then to perform three consecutive steady neck flexion movements using maximum force. Accordingly, the highest measurement can be recorded.

The present method further includes analyzing the results of the subject's assessment data to provide an indicator of muscular function. For example, data such as head circumference and neck length is available through measurement increments incorporated into the head strap and posterior vertical post where vertical adjustment of the force transducer serves to measure the distance between the base of the neck to the occipital tuberosity of the subject's head. For example, the adjustment of the force transducer to be level with the occipital tuberosity of the subject's head further ensures that the force transducer attachment is oriented perpendicular to the moment arm of the neck for calculating the maximum torque associated with a subject's neck flexion strength. For example, the collection of additional data such as age, height and subject weight may also serve to provide a means of normalizing and comparing the data between subjects of similar characteristics. The outcome indicator of the data analysis may be in the range such as poor, good or excellent. The method may further comprise comparing the results of a subject's performance with prior results for the same subject and/or against a collected database of results of subjects to provide a percentile ranking based on the assessment data collected.

The operation of the device may also involve a method of displaying data results on an external device through blue-tooth connection. The obtained results may be fed into a data-processing unit and then provided through an external device that may be connected (hard-wired or wirelessly) to the measurement unit. The external device may comprise but is not limited to a hand-held device or computer. Bluetooth connectivity to an external device may allow the measurements as well as the display of the results to be performed and viewed solely by the subject being tested. This represents a significant advantage over the prior art which requires a practitioner/operator to conduct the measurement as well as to record the measurement of the test.

According to one embodiment, a portable neck strength testing device includes a support member securable to a body of a subject, a measurement unit coupled to the support member and a head mount adjustably connected to the measurement unit. The head mount includes a first portion configured to engage a head of the subject and the head mount is displaceable in response to motion of the first portion. The measurement unit can detect displacement of the head mount and measure therefrom at least a force or a torque exerted on the head mount.

For example, the support member can be safely secured to a subject's body. For example, the support member may be secured at the following locations: the upper body area, the abdominal area, the head, and the shoulders. For example, the manner in which the support member attaches to a wearer's body permits the wearer to maintain normal activity and does not require that the wearer holds the device in place. For example, the support member may be made of a material that allows comfort for the wearer. The material can further allow the support member to safely secured on the subject.

For example, the head mount can have a first portion, a second portion and a central portion between the first and second portion s. The second portion can be adjustably connected to the measurement unit.

The first portion can include a head strap for receiving the head of a subject. For example, the head strap can snuggly encircle the subject's head at the forehead area. The head strap is preferably constructed of elasticized material of a desired width to allow comfort for the subject. The tension of head strap can allow for the movement of the subject's head toward or away from the measurement unit, while at the same time allowing for the lateral mobility of the subject's head.

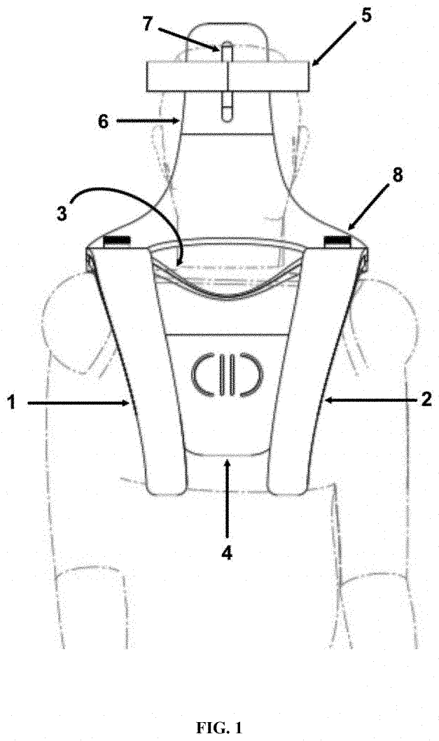

Referring now to FIG. 1, a perspective of a device according to one aspect is shown. The front portion of the device includes two arching legs that overlie on the subject's chest. The right leg (1) and left leg (2) are spaced and configured to allow easy placement of the device on a subject. To ensure user comfort and stable positioning of the device on the subject, padding (3) is provided on the interior surface of the adjustable back plate (4) and front legs (1, 2) of the device. The head strap (5) is adjustable to accommodate various head sizes and the attachment of the force transducer to the posterior vertical post (6) is adjustable through the posterior post slot (7) to accommodate necks of various lengths. The back plate adjustment fastener (8) is utilized to open the device to accommodate positioning of the device on objects of various chest sizes. Once the device is lowered onto the subject, the adjustable back plate is positioned using the back plate adjustment fastener (8) to ensure stability of the device on the subject.

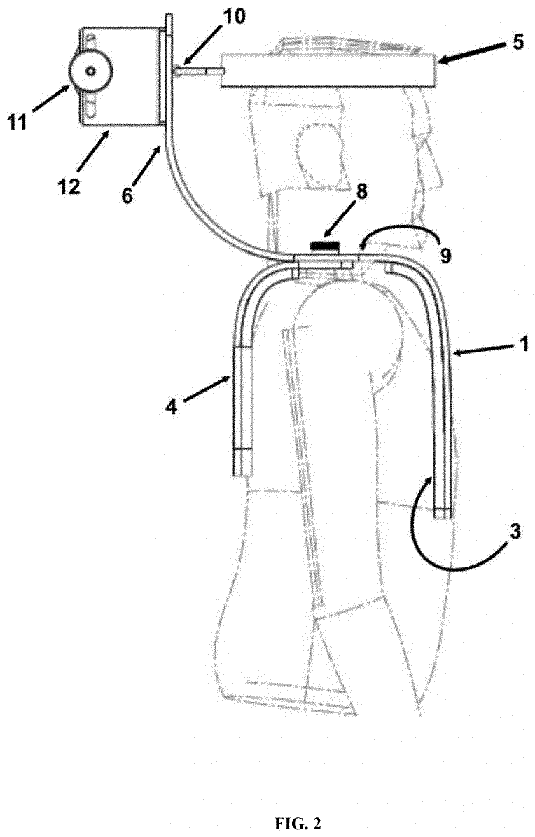

Referring now to FIG. 2, there is shown a side view of the device of FIG. 1. As shown in FIG. 2, the device is positioned on the subject by lowering the device to rest on the shoulders of the subject being assessed.

The portable isometric neck strength testing device can measure the isometric muscle strength of the neck in a human subject's body independent of attachment to a fixed structure (i.e. floor or wall) or a device operator.

The device includes support member having a shoulder section, a (posterior) vertical post, a left leg, a right leg, a back plate and a measurement unit. The shoulder section is connected to the left and right legs.

The left and right legs are used to mount the support member over the chest of the subject. For example, the left leg has a top portion extending horizontally and outwardly from the left side of the posterior vertical post, and a bottom portion extending curvedly and downwardly from the top portion to overlie the chest of the wearer, operable to engage the wearer's chest and shoulder area. Similarly, the right leg can have a top portion extending horizontally and outwardly from the right side of the posterior vertical post, and a bottom portion extending curvedly and downwardly from the top portion to overlie the chest of the wearer, operable to engage the wearer's chest and shoulder area. The inner surface of the legs and the back plate can be lined with padding for comfort when the device is secured to the wearer.

The left and right legs may include an attachment means to secure a removably-attached neck torque measurement assembly. For example, the torque measurement assembly includes two vertically adjustable arms/posts interconnected with a horizontal cross member, wherein the horizontal cross member includes an attachment means for a torque measurement means, wherein the torque measurement means is removably attached to a helmet assembly, operable to secure the wearer's head relative to the torque measurement means.

The adjustable back plate is used to mount the support member over the back of the subject, wherein the back plate is connected to the shoulder section. For example, the back plate can be adjustably connected to the horizontal portions of the right and left legs and is operable to secure the right and left legs against the chest and shoulder area of the wearer. Furthermore, the back plate can be adjustably attached to both sides of the shoulder section of the device using back plate adjustment fasteners operable to allow movement of the back plate horizontally in a backward and forward direction with respect to the human subject's body for the purpose of securing the device to the wearer.

A back plate adjustment fastener is located on the shoulder section. It is used to adjust the back plate on the back of the subject to secure and stabilize the device on the upper body of the subject.

The vertical post is connected to the shoulder section and extends vertically away from the back plate. The vertical post can be positioned opposite to the direction of the force applied by the wearer's neck muscles. The vertical post may be adjustable to allow rotation of the post to measure the quantity of force applied by the neck in directions incremented from 0-360° and include front flexion, back extension, and lateral flexion.

The right leg (1), as shown in FIGS. 1-2, and the left leg (2), as shown in FIG. 1 and the adjustable back plate (4) are lined with padding (3) for comfort purposes. The legs extend horizontally from each respective side of the base of the posterior vertical post (6) over the shoulder section (9) of the wearer and then arch down to overlie the wearer's chest. The adjustable back plate (4) is positioned by adjusting the back plate adjustment fastener (8), or other suitable adjusting tool, to secure the device to the subject. The head strap (5) and measurement unit including a force transducer (10) are integrated into the posterior vertical post (6) using an attachment member (11). To ensure procedural consistency in force measurements, the attachment member (11) may be adjusted vertically on the anchoring bracket (12) to be aligned with the occipital tuberosity of the wearer's head.

For example, the attachment member has a screwed member which includes a collared bolt and a nut, wherein the measurement unit is pivotally fitted over the collared bolt and screwed to the anchoring bracket through the bracket slot so as to allow a means for selectively adjusting the height of the measurement unit. The posterior vertical post can include a slot, wherein the slot is operable to allow horizontal alignment of the force transducer and the head strap to a reference point which includes but is not limited to the occipital tuberosity of the wearer's head.

The posterior vertical post may include measurement increment markings operable to allow measurement of neck length from the base of the neck to reference point including but not limited to the occipital tuberosity of the head.

The head strap may further include measurement markings operable to record head circumference of the subject being tested.

The measurement unit may further include a force transducer capable of displaying measurement results and of transferring data to an external processor or device through hardwire and/or wireless connection.

The device may further include a harness assembly operable to counterbalance and measure the force exerted by a test subject's body member.

A typical movement for assessing the neck flexion strength is a movement of the chin to chest in a forceful manner in order to have the device record the maximum neck flexion strength of the subject. For example, the subject is instructed to maintain an upright posture during the assessment. When the device is secured through the adjustment of the back plate and when the subject is instructed to flex the neck, the right leg (1) and the left leg (2) of the device engage the chest and shoulder area of the subject which act to stabilize the device to isolate the neck muscles as a sole means of flexing the neck. For example, the support member can function as a lever where the head is positioned at the fulcrum and chest and posterior vertical post serves as the load and effort arms of the lever respectively. When the subject flexes forward, the muscles themselves create the load and allow isometric neck strength to be measured.

For example, the lever does not strain the muscles, but the muscles themselves create the load and allow the isometric neck muscle strength to be measured. A display on the measuring unit (or the force transducer) shows measurement data and may also provide a person skilled in the art or a subject being tested with additional useful information (such as neck circumference, head circumference, neck length, age, gender, etc.).

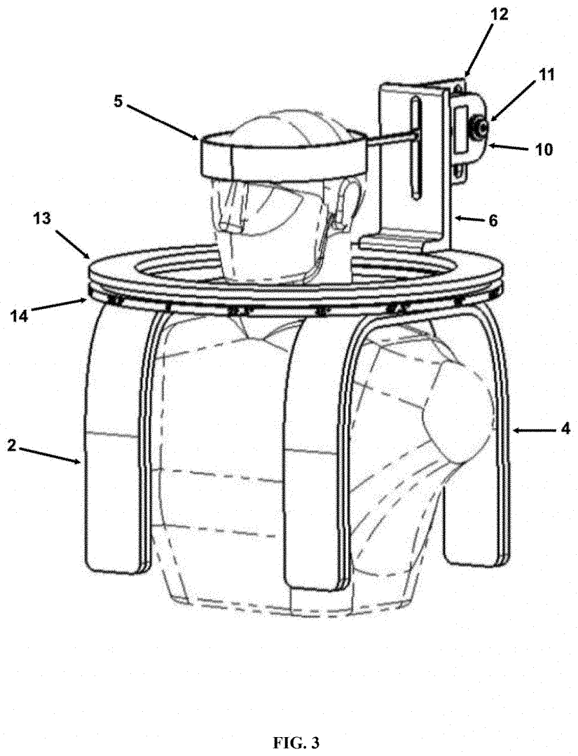

In FIG. 3, an embodiment of a variant design of the present subject matter includes a rotating turntable (13) and a turntable base (14) as part of to the shoulder section of the device. In this embodiment, the front legs and the back plate are mounted on the turntable base, and the vertical post is mounted on the rotating turntable. Further, the rotating turntable is rotatable with respect to the turntable base to allow force measurements to be recorded through a 360-degree range.

It will be apparent to one with skill in the art that the variant design will provide a portable neck strength measurement device (without the need for external support or attachment) that allows measurement of force in various directions that include but are not limited to front flexion, back extension, lateral right and left flexion as well as incremental degrees throughout the range of 360 degrees defined by rotation of the rotating turntable with respect to the turntable base.

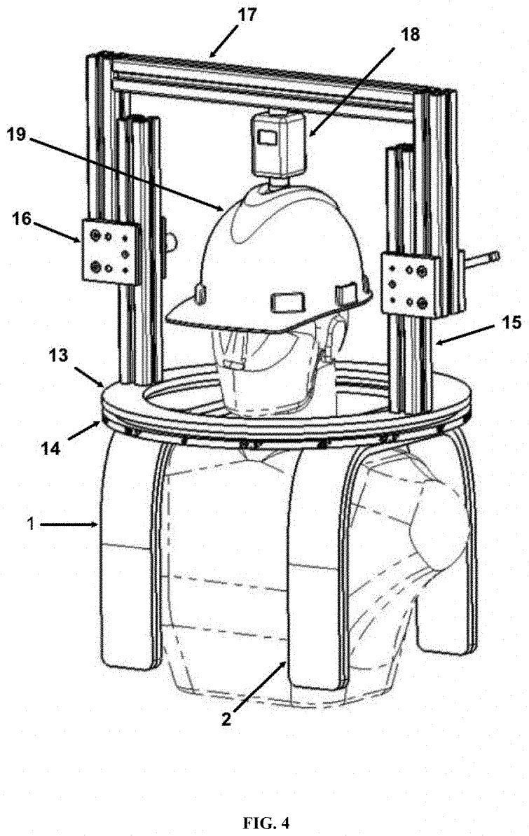

An additional embodiment of the present subject matter, as shown in FIG. 4, presents a removably-connected torque measurement assembly. The torque measuring assembly includes a pair of arms extending away from the shoulder section, an overhead cross member mounted on the pair of arms, and a rotational torque measuring unit mounted on the overhead cross member and configured to engage the head of the subject for measuring the torque in response to rotation of the head. For example, the torque measuring unit can include a torque sensor (i.e. torque transducer or torque meter) for measuring and recording the torque on when the assembly is rotated. An example of torque sensors is the Eastwood Electronic Torque Adapter™.

The torque measuring unit can include a processor to transmit, via Bluetooth connectivity, collected and processed data to an external device, such as a smartphone, a personal computer or a server. For example, the torque measuring unit can have an input interface (e.g. a keyboard) that allows a user to enter data such as neck circumference, head circumference, neck length, age, gender. In addition to torque data recorded by the measuring unit, the processor may use the user input data to compute the strength of the neck muscles of a subject in response to rotation of the head of the subject. For example, torque data recorded by the measuring unit and/or user input data may be compared to historical data (e.g. data collected for other subjects and/or circumstances pertaining to a particular subject) to establish a baseline for neck strength measurements for improving accuracy of the measurements.

The torque measuring unit can also have a screen or any other type of output interfaces to display collected or processed data.

As shown in FIG. 4, the vertical posts of the assembly (15) are attached to the shoulder section of the right (1) and left (2) legs using a fastening means to the device. The torque measurement assembly measures rotational torque of the neck, and includes a vertical post height adjustment control means (16) to vertically adjust the overhead cross member (17) with the cross member accepting a rotational torque measuring unit (18) to measure the torque of a rotating head.

The member of the torque measuring means is coupled to the overhead cross member at a first axial position and a second member of the torque measuring means is coupled fixed to a second axial position on a removable helmet assembly (19) that fixes the head in position relative to the helmet.

Rotation of the head allows torque measurements to be recorded as the subject rotates the head to the right or left. It will be apparent to one with skill in the art that the removable assembly will provide a portable neck strength measurement device (without the need for practitioner connection or external support or attachment) that allows the measurement of torque as the head is rotated to the left or right.

The devices as described in the present disclosure can measure neck flexion strength in a forward direction. Further, they may be used for other neck strength measurements. In particular, the embodiments of the present subject matter may also be used to measure neck extension strength by simply rotating the device 180°. Expressions such as “flexion” and “measuring”, used herein should not be taken as to limit the scope of the present subject matter and includes all other kinds of measurements or exercises with which the present subject matter could be used and may be useful, as apparent to a person skilled in the art. For example, a variant design of the present subject matter, as shown in FIG. 3, provides a rotating turntable and turntable base that is fixed to the shoulder portion of the device. Rotation of the turntable allows the measurement of flexion force through a range of 0-360°. Furthermore, the addition of a removably connected torque measuring assembly, as shown in FIG. 4 allows for measurement of neck rotation torque in either the left or right direction.

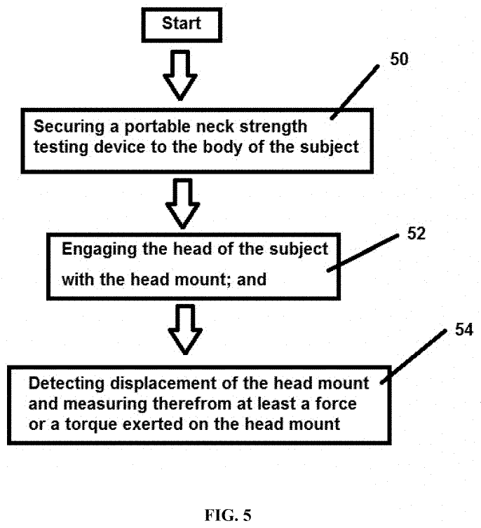

FIG. 5 shows a method for measuring a force exerted by neck muscles of a subject. The method includes at 50 securing a portable neck strength testing device to a body of a subject. The device can include a support member securable to a body of a subject, a measurement unit coupled to the support member and a head mount adjustably connected to the measurement unit. At 52, the method includes engaging the head of the subject with the head mount. At 54, the method includes detecting, with the measurement unit, displacement of the head mount and measuring therefrom at least a force or a torque exerted on the head mount

For example, securing the device to the subject can include mounting one or more front legs of the device over the chest of the subject, mounting an adjustable back plate of the device over the back of the subject, and adjusting, with a back plate adjustment fastener located on the shoulder section of the device, the back plate on the back of the subject to secure and stabilize the device on the upper body of the subject.

The method can further include measuring a force applied by neck muscles of the subject to the head mount with a force transducer connected to the head mount.

The method can further include adjusting the force transducer on a vertical post connected to the shoulder section and extending vertically away from the back plate such that the force transducer and the head mount are positionable to align with the occipital tuberosity of the head of the subject.

The method can further include measuring with the force transducer neck flexion strength relative to the vertical post from motion of the head mount when the head mount engages the head of the subject.

The method can further include measuring with the force transducer a maximum rotational torque applied by neck muscles of the subject when the head is turned to the left or right directions.

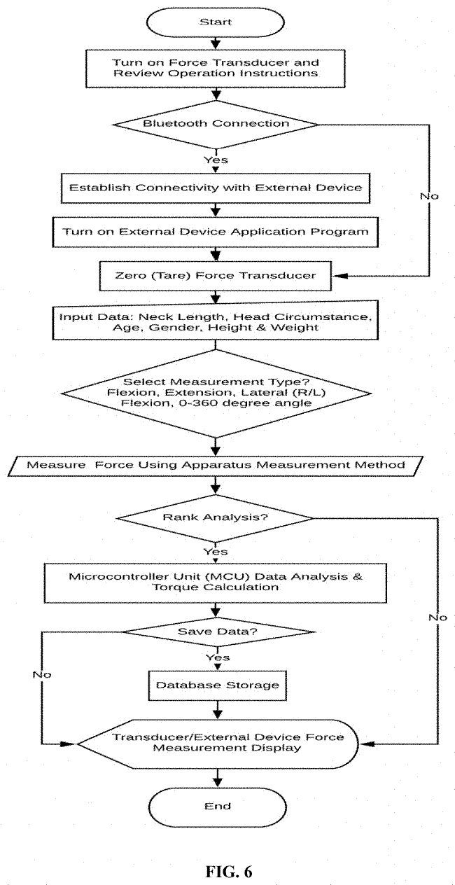

FIG. 6 shows a flow chart of a method for measuring neck strength. The method can be processed by processor in communication with a force traducer or a torque sensor as previously described. The processed data can be viewed either on a measuring unit/device that includes the processor or on an external device that is connected to the processor via Bluetooth™.

For example, an external device can establish an electronic communication (e.g. Bluetooth™) with a processor connected to the force or torque transducer to detect sensor state, select measurement types (e.g. flexion, extension, lateral flexion, degree angle, etc.) and gather measurement data. A user can also enter measurements subject parameters (e.g. neck length, head size, age, gender, height and weight) into the computer program interface with the processor.

The processor can be configured to analyze data collected by the force and/or torque sensors and/or user input data. Based on the analysis, the processor can further determine information that acts as an indicator of risk for head and/or neck injury based on the data analysis.

Embodiments of the of the portable device for measuring neck strength as described in the present disclosure are an asset in sport programs designed to reduce the incidence of concussions and head injuries. Such programs involving neck strength flexion testing would consist of pre- and post-activity measurements of neck flexion strength as well as testing to measure the progress of neck strengthening exercises.

It will be apparent to one with skill in the art that the portable isometric neck strength testing device may be provided with some or all of the mentioned features and components without departing from the scope of the present disclosure

The information disclosed herein is provided merely to illustrate principles and should not be construed as limiting the scope of the subject matter of the terms of the claims. The written specification and figures are, accordingly, to be regarded in an illustrative rather than a restrictive sense. Moreover, the principles disclosed may be applied to achieve the advantages described herein and to achieve other advantages or to satisfy other objectives, as well.

1. A portable neck strength testing device, comprising:
a support member securable to a body of a subject;
a measurement unit coupled to the support member;
a head mount adjustably connected to the measurement unit,
wherein the head mount comprises a first portion configured to engage a head of the subject;
wherein the head mount is displaceable in response to motion of the first portion; and
wherein the measurement unit is configured to detect displacement of the head mount and measure therefrom at least a force or a torque exerted on the head mount.

2. The device of any one of the preceding claims, wherein the support member comprises:
one or more front legs for mounting the support member over the chest of the subject;
a shoulder section connected to the one or more front legs;
an adjustable back plate for mounting the support member over the back of the subject, wherein the back plate is connected to the shoulder section;
a back plate adjustment fastener located on the shoulder section and configured to adjust the back plate on the back of the subject to secure and stabilize the device on the upper body of the subject; and
a vertical post connected to the shoulder section and extending vertically away from the back plate.

3. The device of any one of the preceding claims, wherein the shoulder section comprises a turntable and a base, the front legs and the back plate are mounted on the base, the post is mounted on the turntable, and the turntable is rotatable with respect to the base.

4. The device of any one of the preceding claims, further comprising a removable torque measuring assembly for measuring neck rotation torque in either a left or right direction, wherein the torque measuring assembly comprises:
a pair of arms extending away from the shoulder section,
an overhead cross member mounted on the pair of arms, and
a rotational torque measuring unit mounted on the overhead cross member and configured to engage the head of the subject for measuring the torque in response to rotation of the head.

5. The device of any one of the preceding claims further comprising an anchoring bracket mounted on the vertical post to allow vertical adjustment of the measurement unit.

6. The device of any one of the preceding claims, wherein the measurement unit comprises a force transducer, and wherein the head mount is adjustably connected to the force transducer configured to detect and measure a force exerted on the head mount.

7. The device of any one of the preceding claims, wherein the force transducer is adjustable on the vertical post such that the force transducer and the head mount are positionable to align with the occipital tuberosity of the head of the subject.

8. The device of any one of the preceding claims, wherein the force transducer is configured to measure neck flexion strength relative to the vertical post from motion of the head mount when the head mount engages the head of the subject.

9. The device of any one of the preceding claims, wherein the head mount is displaceable in response to various motions of the first portion or the head mount, including front flexion, lateral flexion, extension, or a direction defined within a range of 0-360°.

10. The device of any one of the preceding claims, wherein the measurement unit is configured to measure a maximum rotational torque applied by neck muscles of a subject when the head is turned to the left or right directions.

11. The device of any one of the preceding claims, wherein the support member comprises a material including aluminum, plastic or carbon fiber.

12. A method for measuring a force exerted by neck muscles of a subject, the method comprising:
providing a portable neck strength testing device, the device comprising a support member securable to a body of a subject, a measurement unit coupled to the support member and a head mount adjustably connected to the measurement unit;
engaging the head of the subject with the head mount; and
detecting, with the measurement unit, displacement of the head mount and measuring therefrom at least a force or a torque exerted on the head mount

13. The method of the preceding claim wherein securing the device to the subject comprises:
mounting one or more front legs of the device over the chest of the subject, the one or more legs being connected to a shoulder section of the device;
mounting an adjustable back plate of the device over the back of the subject, the back plate being connected to the shoulder section of the device; and
adjusting, with a back plate adjustment fastener located on the shoulder section of the device, the back plate on the back of the subject to secure and stabilize the device on the upper body of the subject.

14. The method of the preceding claims further comprising measuring a force applied by neck muscles of the subject to the head mount with a force transducer connected to the head mount.

15. The method of the preceding claims further comprising adjusting the force transducer on a vertical post connected to the shoulder section and extending vertically away from the back plate such that the force transducer and the head mount are positionable to align with the occipital tuberosity of the head of the subject.

16. The method of the preceding claims further comprising measuring with the force transducer neck flexion strength relative to the vertical post from motion of the head mount when the head mount engages the head of the subject.

17. The method of the preceding claims further comprising measuring with the force transducer a maximum rotational torque applied by neck muscles of the subject when the head is turned to the left or right directions.
